(19) United States
(12) Patent Application Publication
(10) Pub. No.: US 2009/0035288 A1
(43) Pub. Date: Feb. 5, 2009

(54) EDIBLE PRODUCT CONTAINING BENEFICIAL BACTERIA

(76) Inventors: Ruud Albers, Vlaardingen (NL); Stanley Brul, Vlaardingen (NL); Adrianus Marinus Ledeboer, Vlaardingen (NL); Willem Maarten Meijer, Vlaardingen (NL)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/989,059

(22) PCT Filed: Jun. 29, 2006
(86) PCT No.: PCT/EP2006/006306

§ 371 Date: Jan. 18, 2008

(30) Foreign Application Priority Data
Jul. 20, 2005 (EP) 05076666.6

(51) Int. Cl. A61K 35/74

(52) U.S. Cl. 424/93.45; 424/93.4

(57) ABSTRACT

The present invention provides a method of preparing an edible product comprising non-viable bacteria providing a health benefit, the method comprising subjecting viable bacteria to at least two sub-lethal treatments to obtain the non-viable bacteria providing a health benefit, each sub-lethal treatment on its own not being sufficient to render the bacteria non-viable. The method porvides non-viable bacteria providing health benefits but which can conveniently be incorporated into a range of edible products.

### FIELD OF INVENTION

The present invention relates to edible products, especially food and beverage products, comprising bacteria which are non-viable but which when administered in suitable amounts provide a beneficial effect, particularly a health benefit, to the subject consuming them. In particular the invention relates to said edible products which comprise such bacteria which have been subjected to two or more sub-lethal treatments to provide the bacteria in a non-viable form but which are still able to provide the aforementioned health benefits.

### BACKGROUND OF THE INVENTION

The application of probiotic bacteria in food products is often associated with health effects, see for example A. C. Ouwehand et al. in Int. Dairy Journal 8 (1998) 749-758. In particular the application of probiotic bacteria is associated with health effects for example relating to the gut well-being such as IBS (Irritable Bowel Syndrome), IBD (inflammatory bowel diseases), reduction of lactose maldigestion, clinical symptoms of diarrhea, immune stimulation, anti-tumor activity and enhancement of mineral uptake. It is generally believed that some of the health effects of probiotic bacteria are related to their immunomodulatory and anti-inflammatory properties at mucosal sites. These health effects are most likely initiated by effects of the probiotic bacteria on the mucosal immune system in the ileum and jejunum. Said modulatory effects of probiotic bacteria have been demonstrated to beneficially affect e.g. resistance to infections, allergic diseases and inflammatory bowel diseases.

It is generally recognised in the art that the inclusion of certain bacteria in edible products, such as food products, is desirable to provide health benefits upon the consumption of the edible product. For example WO 94/00019 describes the addition of desirable viable lactic acid bacteria to baked products for health purposes.

Traditionally probiotic bacteria have been employed as viable microorganisms as it was believed that the bacteria must be in a viable state for them to provide their beneficial health effects.

However, using only viable probiotic bacteria has the disadvantage that their use is limited to edible products having product characteristics which are suitable for viable bacteria and which are produced by processing techniques that are suitable for viable bacteria. This means that edible products comprising viable bacteria are expensive to prepare and that the methods of storing the viable probiotic bacteria and the foods comprising them is complicated and hence further increase the costs of the edible products.

Furthermore, a problem with the use of viable probiotic bacteria in edible products is it that the formulation of the product often needs to be adapted to ensure that the viable character of the probiotic bacteria can be maintained. For example, low or high pH values for the edible product may not be suitable, high mineral contents may not be possible and/or the product may need a minimum water activity. This limits the formulation flexibility of the edible products which is undesirable.

Another possible problem with the use of viable probiotic bacteria in edible products is that often the products will require storage at relative low temperatures to ensure that they are not fermented by the bacteria. If the fermentation process proceeds this may lead to products which are either too acid or which have unwanted organoleptic properties, such as poor physical structure and/or poor taste, due to so-called post-acidification.

It has been suggested that probiotic bacteria do not need to be in a viable state in order to confer at least some of their probiotic effects to a subject consuming them. For example, A. C. Ouwehand et al. in Int. Dairy Journal 8 (1998) 749-758 discuss the health effects of cultured milk products with viable and non-viable bacteria.

WO 2004/069156 discloses formulations comprising inactivated probiotic bacteria. The bacteria are inactivated by irradiation treatments. The paper by the same inventors “Toll-like receptors 9 signalling mediates the anti-inflammatory effects of probiotics in murine experimental colitis” by Rachmilewitz et al Gastroenterology 2004; 126: 520-528 discloses what the inventors believe to be the theory behind the inactivation and the remaining probiotic effect.

The Rachmilewitz references indicate that some of the important immunomodulatory and anti-inflammatory properties of viable probiotic micro-organisms may be retained in non-viable micro-organisms if they are rendered non-viable under certain conditions. These references suggest that when micro-organisms that are rendered non-viable in conventional ways such as pasteurization or sterilization their structural integrity is impaired which results in the rapid disintegration of the non-viable micro-organisms in the proximal parts of the intestinal tract. In contrast, the non-viable micro-organisms of Rachmilewitz which are rendered non-viable by the use of gamma radiation are said to retain their integrity in the proximal intestinal tract which enables the interaction of particular microbial patterns, in this case unmethylated DNA, with Toll-like receptors on the mucosal immune system. Such interactions are then described to result in the described immunomodulating and anti-inflammatory effects.

WO 01/95741 describes the use of non-viable Lactobacillus bacteria in food products. The Lactobacillus bacteria are rendered non-viable by the application of a single processing step and prevent the food product undergoing further fermentation from the presence of the bacteria.

Whilst it is suggested in the art that certain non-viable bacteria may give at least some useful health benefits, to date the bacteria have been rendered non-viable by a single processing step.

The use of such a single processing step may have one or more of the following disadvantages;
the single processing step can be difficult to control so that it is not always possible to ensure that the population is rendered non-viable whilst maintaining the structural integrity of the bacteria,
different processing conditions may be applied across the whole of a food product or across a batch of such products so that treatment is ineffective or irregular,
harsh single step processing conditions may be detrimental to the food product itself,
the conditions used may limit the flexibility of the food formulation or the processing conditions as the single processing step needs to achieve conditions which are harsh enough to render the bacteria non-viable,
where irradiation is used as the single processing step this generally has low consumer acceptability or it may not be widely allowed or accepted in different regions.

The present invention seeks to address one or more of the above problems.

In particular, the present invention seeks to provide a convenient and effective method of providing an edible product comprising non-viable bacteria providing health benefits. In particular, the invention seeks to provide a method which can be used to prepare a wide variety of edible products comprising the aforementioned types of bacteria.

### SUMMARY OF THE INVENTION

Surprisingly it has been found that when at least two treatments are used on the bacteria, each treatment on its own not being sufficient to render the bacteria non-viable, the bacteria are rendered non-viable by the combined treatment but are still able to provide health benefits to the person consuming the bacteria.

Thus according to a first aspect the present invention provides a method of preparing an edible product comprising non-viable bacteria providing a health benefit to the subject consuming the bacteria, wherein the method comprises subjecting viable bacteria to at least two sub-lethal treatments to obtain the non-viable bacteria providing a health benefit.

It is preferred that the edible product is a food or beverage product. It is preferred that the health benefit is a probiotic effect.

It is further preferred that the bacteria providing said health benefit are non-pathogenic bacteria. It is further preferred that the bacteria providing said health benefit are substantially structurally intact in the edible product. It is further preferred that the bacteria retain conserved microbial patterns that can be recognized by pattern recognition receptors of the immune system, preferably that the conserved microbial patterns comprise DNA and/or cell wall constituents.

It is further preferred that the bacteria are selected from the genera Lactobacillus or Bifidobacterium.

Preferably the edible product contains between 10⁶ and 10¹¹ bacteria per serving.

According to one embodiment of the invention the manufacturer of the edible product could carry out the first sub-lethal treatment and the consumer of the edible product could carry out the second sub-lethal treatment prior to consumption of the product.

The present invention provides several advantages including that the bacteria are rendered non-viable but remain substantially structurally intact and retain their ability to modulate immune function and inflammatory responses. This maximises the retention of the health benefits from the bacteria. One or more of the following advantages may also be obtained according to the present invention;
the two sub-lethal treatments could be carried out at different times during the preparation of the edible product as required and/or by different operators e.g. one by the edible product manufacturer and one by the product consumer,
the use of at least two sub-lethal treatments provides for flexibility in the preparation of the edible product as it is not necessary to use a single harsh treatment. This allows for different steps to be chosen dependent upon the type of edible product and such steps may often be chosen from conventional processing techniques. Furthermore, this may provide better sensory and nutritional properties for edible products.
as different sub-lethal treatments may be combined to render the bacteria non-viable, it is not necessary to rely solely on treatments which have generally low consumer acceptance such as irradiation.

Thus according to a second aspect the present invention provides an edible product obtainable according to the first aspect of the invention

Preferably the edible product is a food or beverage product.

“Probiotic bacteria”, as used herein, means bacteria which when administered in adequate amounts confer a health benefit to the consumer thereof.

By the term “health-benefit” as used herein is meant improving or maintaining at least one aspect of the health of an individual.

By the term “non-viable bacteria” as used herein is meant a population of bacteria that is not capable of replicating under any known conditions. However, it is to be understood that due to normal biological variations in a population, a small percentage of the population (i.e. 5% or less) may still be viable and thus capable of replication under suitable growing conditions in a population which is otherwise defined as non-viable. The percentage of a population that is viable can be determined with the help of bacteria count methods well-known in the art (see Examples). These methods preferably employ growing conditions (growth medium, temperature etc.) that are optimal for growth of the bacteria tested.

By the term “viable bacteria” as used herein is meant a population of bacteria that is capable of replicating under suitable conditions under which replication is possible.

However, it is to be understood that due to normal biological variations in a population, a small percentage of the population (i.e. 5% or less) may still be non-viable and thus not capable of replication under those conditions in a population which is otherwise defined as viable.

By the term “contacting” as used herein is meant that the bacteria and the edible product or at least one ingredient thereof are brought into direct contact with each other by any suitable means.

By the term “sub-lethal treatment” as used herein is meant a treatment under which a population of bacteria is damaged but has not fully lost its replication capacity as a population so that this is at least in part retained or can be regained under suitable growth conditions for that type of bacteria.

The combination of two or more sub-lethal treatments according to the invention results in at least 95% of the bacteria population being rendered non-viable. Preferably, the aforementioned combination of sub-lethal treatments results in the bacteria population being rendered non-viable.

By the term “suitable growth conditions” as used herein is meant the conditions for a given bacterial strain under which that bacteria strain will replicate and refer to a combination of pH, medium and temperature where normally a diluted version of said strain in viable form (say about 10⁶ bacteria per gram) would grow to a density of at least 10⁸bacteria per gram within a normal period of growth.

By the term “pathogenic bacteria” as used herein is meant bacteria that are capable of causing an infection in an immunocompetent host, or, that are capable of intoxicating such host under suitable conditions.

By the term “non-pathogenic bacteria” as used herein is meant bacteria that is not capable of causing an infection in an immunocompetent host, or, that are not capable of intoxicating such host under suitable conditions.

By the term “substantially structurally intact” as used herein is meant non-viable bacteria which are still sufficiently intact to avoid or delay disintegration in the distal intestinal tract thereby enabling the interaction of (conserved structures of) the non-viable bacteria with the immune system, particular the mucosal immune system.

By the term “per serving” as used herein is meant the amount of a given edible product, and especially a food or beverage product, that is intended to be, or is packaged so as to be, consumed in a single sitting. Therefore, the product may also be packaged as multiple serving portions.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material or conditions of reaction, physical properties of materials and/or use are to be understood as modified by the word “about.” All amounts are by weight, based on the total weight of the relevant product, unless otherwise specified.

Unless stated otherwise, all percentages are by weight based on the total weight of the composition.

For a more complete explanation of the above and other features and advantages of the invention, reference should be made to the following description of the preferred embodiments. The preferred embodiments apply to all aspects of the invention and can be used as appropriate for each aspect unless the context requires otherwise.

### DETAILED DESCRIPTION OF THE INVENTION

### Sub-Lethal Treatments

According to the present invention, the bacteria which provide a health benefit to the subject consuming the bacteria are subjected to at least two sub-lethal treatments during the preparation of an edible product, each sub-lethal treatment on its own not being sufficient to render the bacteria non-viable. These treatments may occur prior to incorporation of the bacteria in the edible product, e.g. by treating the bacteria or a mixture of the bacteria and one or more food ingredients. Likewise, it is possible to subject the bacteria to sub-lethal treatment during different stages of the preparation process, e.g. by first treating the bacteria and subsequently treating the edible product containing the treated bacteria.

Any suitable sub-lethal treatment may be used according to the present invention. The references “Basic aspects of food preservation by hurdle technology” by Leistner., L. Int Journal of Food Microbiology 55 (2000) 181-186 and “Combined methods for food preservation” by Leistner., L. 1999 in Handbook of Food Preservation, Shafiur Rahman., M. (Ed.) Marcel Dekker, New York, 457-485 disclose suitable sub-lethal treatments which may be used and are incorporated by reference herein.

Typically, the present method employs at least two sub-lethal treatments, wherein at least one sub-lethal treatment, when applied as a single treatment of the viable bacteria, reduces the replication capacity of said viable bacteria by at least 5%. Accordingly, the present method advantageously comprises subjecting viable bacteria to at least two sub-lethal treatments, at least one of which sub-lethal treatments is capable of reducing the replication capacity of the (original) viable bacteria by at least 5%, preferably by at least 10%. The replication capacity of a bacteria population is suitably determined by a bacteria count method as mentioned herein before.

The inventors have unexpectedly found that bacteria can be rendered non-viable effectively by subjecting them to a sub-lethal treatment that hardly (or not) affects replication capacity and another sub-lethal treatment that reduces the replication capacity of the (original) viable bacteria by at least 5% (e.g. less 5-50%). In particular it was found that the bacteria may be rendered non-viable by combining a low pH that in itself hardly affects replication capacity with another sub-lethal treatment that is capable of reducing the replication capacity by at least 5%. Preferably, these sub-lethal treatments occur at least partially simultaneously.

According to a particularly preferred embodiment, the present method employs at least two sub-lethal treatments that each on its own is capable of reducing the replication capacity of the (original) viable bacteria by at least 5%, preferably by at least 10%.

In another preferred embodiment, the present method employs two or more sub-lethal treatments that each on its own reduces the replication capacity of the viable bacteria by not more than 60%, preferably by not more than 50%. In another preferred embodiment, the method utilizes two or more sub-lethal treatments, wherein the sum of the percentages reduction in replication capacity observed for each sub-lethal treatment does not exceed 60%, more preferably does not exceed 50%. An example of a method meeting this requirement is a method that employs one sub-lethal treatment that in itself yields a reduction in replication capacity of e.g. 10% and another sub-lethal treatment which per se yields a reduction in replication capacity of e.g. 5%. Whereas the sum of the percentages reduction in replication capacity for these two sub-lethal treatment is only 15%, the combination of said treatments in accordance with the present invention yields an overall reduction in replication capacity of, for instance, 95% or more.

According to another advantageous embodiment of the invention, the present method comprises either:
a) subjecting viable bacteria providing said health benefit to at least two sub-lethal treatments and subsequently contacting the non-viable bacteria thereby produced with an edible product or at least one ingredient thereof, or
b) contacting viable bacteria providing said health benefit with an edible product and subsequently subjecting the edible product comprising the viable bacteria to at least two sub-lethal treatments, or
c) contacting viable bacteria providing said health benefit with at least one ingredient of an edible product and subsequently subjecting the mixture of the viable bacteria and the ingredient to at least two sub-lethal treatments.

It is preferred that each of the two or more sub-lethal treatment steps is independently selected from;
(i) the application of pressure
(ii) adjusting the pH
(iii) adjusting the osmotic pressure
(iv) heating
(v) homogenisation
(vi) freeze-thaw cycles
(vii) spray-drying
(viii) adding one or more agents having a bactericidal effect
(ix) applying a pulsed electric field

Alternative suitable conditions for carrying out each of the sub-lethal steps will be known to the person skilled in the art. It is preferred that the sub-lethal treatments are independently selected from the following;
(i) Applying a pressure of from 150 Mpa to 400 Mpa at from −30° C. to 25° C. for between 20 to 60 seconds,
(ii) Adjusting the pH to in the range of from pH 3 to 5, preferably from pH 4 to 5 or from pH8 to 9,
(iii) Adjusting the osmotic pressure by adding a suitable amount of an alkaline or alkaline earth metal salt,
(iv) Heating to a temperature of from 10° C. to 25° C., preferably from 10° C. to 15° C. above the optimal growing temperature for the bacteria for between 1 to 5 minute
(v) Homogenising at from 20 to 30 bar at 10° C. to 15° C. above the optimal growing temperature for the bacteria for between 1 to 5 minutes
(vi) Subjecting to a freezing step and subsequent thawing step for between 5 to 25 cycles.
(vii) Adding a suitable amount of one or more agent(s) that have a bactericidal effect and which are chosen from sodium sorbate, lysozym and nisin.
(viii) Applying a pulsed electric field using between 15 to 100 kV/cm with a pulse length of between 1 to 10 μs at from 10° C. to 50° C.

Another sub-lethal treatment could be the use of irradiation provided that the radiation treatment was controlled such that a sub-lethal result was obtained. Suitable conditions for a sub-lethal irradiation treatment include irradiating at from 0.1 to 1 megarad, using a ¹³⁷CS source at a rate of 8 Gy/min overnight. However, it is preferred that the sub-lethal treatments according to the invention do not include more than one sub-lethal irradiation treatment.

### Beneficial Bacteria

Any bacteria which provides a health benefit to the subject consuming the bacteria may be used according to the invention. These beneficial effects preferably include immuno modulatory and anti-inflammatory properties.

It is preferred according to the invention that the health benefit is a probiotic effect and thus that the bacteria are probiotic bacteria. It is further preferred that the bacteria are non-pathogenic bacteria.

The probiotic bacteria used according to the present invention may be any conventional probiotic bacteria. It is preferred that the probiotic bacteria are selected from genera Bifidobacterium, Propionibacterium, Enterococcus, Streptococcus, Lactococcus, Bacillus, Pediococcus, Micrococcus, Leuconostoc, Weissella, Oenococcus and Lactobacillus, with Lactobacillus and Bifidobacterium being the most preferred.

Suitable types of probiotic bacteria which may be used include; Bacillus natto, Bifidobacterium adolescentis, B. animalis, B. breve, B. bifidum, B. infantis, B. lactis, B. longum, Enterococcus faecium, Enterococcus faecalis, Escherichia coli, Lactobacillus acidophilus, L. brevis, L. casei, L. delbrueckii, L. fermentum, L. gasseri, L. helveticus, L. johnsonii, L. lactis, L. paracasei, L. plantarum, L. reuteri, L. rhamnosus, L. sakei, L. salivarius, Lactococcus lactis, Lactococcus cremoris, Leuconostoc mesenteroides, Leuconostoc lactis, Pediococcus acidilactici, P. cerevisiae, P. pentosaceus, Propionibacterium freudenreichii, Propionibacterium shermanii and Streptococcus salivarius.

Particular probiotic strains which are suitable according to the present invention are: Lactobacillus casei shirota, Lactobacillus casei immunitas, Lactobacillus casei DN-114 001, Lactobacillus rhamnosus GG (ATCC53103), Lactobacillus reuteri ATCC55730/SD2112, Lactobacillus rhamnosus HN001, Lactobacillus plantarum 299v (DSM9843), Lactobacillus johnsonii La1 (I-1225 CNCM), Lactobacillus plantarum WCFS1, Lactobacillus helveticus CP53, Bifidobacterium lactis HN019, Bifidobacterium animalis DN-173010, Bifidobacterium animalis Bb12, Bifidobacterium infantis 35624, Lactobacillus casei 431, Lactobacillus acidophilus NCFM, Lactobacillus reuteri ING1, Lactobacillus salivarius UCC118, Propionibacterium freudenreichii JS, Escherichia coli Nissle 1917.

It is to be understood that any of the above mentioned bacteria may be genetically modified bacteria or they may be food-grade bacteria commonly used in industrial processes.

Advantageously the amount of non-viable bacteria providing a health benefit (to the subject consuming the bacteria) in the edible product are from 10⁶ and 10¹¹ per serving, more preferred from 10⁷ to 10¹⁰ per serving most preferred 10⁸ to 10¹⁰ per serving or per 100 g of the product. Serving sizes of various products are given in Table 1.

The bacteria used according to the invention may according to one embodiment be bacteria which have been salvaged from the waste stream of another food processing operation.

The bacteria may be contacted with the edible product or one or more of its ingredients by any suitable means, e.g. mixing therewith or being applied as a coating thereto either alone or with another ingredient e.g. as a solution. For example in the process of making a bakery product the non-viable bacteria may be added to the dough, followed by baking the dough in the oven to prepare the final product. In another example non-viable bacteria may be added to a ice-premix followed by (optional) heat treatment and freezing to produce a frozen dessert. Alternatively, and especially where the bacteria have been rendered non-viable prior to contacting with the edible product or an ingredient thereof, the bacteria may be contacted with the product/ingredient by means of suitable packaging. This may be achieved for example by having the non-viable bacteria present on a part of the product packaging (such as a straw or container lid) so that the product/ingredient contacts the non-viable bacteria upon egress of the product from the packaging.

Beneficial Effects from the Bacteria

The non-viable bacteria according to the present invention are sufficiently intact to avoid or delay disintegration in the distal intestinal tract thereby enabling the interaction of their so-called conserved microbial patterns such as cell wall constituents such as lipopolysaccharides, lipoteichoic acid, peptidoglycans, and unmethylated DNA, with so-called pattern-recognition receptors of the (mucosal) immune system such as Toll-like receptors and Nod receptors. These interactions can result in beneficial modulation of immune function which could result in e.g. increased resistance to infections, suppression of inflammatory responses and alleviation or prevention of allergies or auto-immune diseases. An explanation of the role of Toll-like receptors is given in the reference Adv Exp Med Boil. 2005; 560:11-8 by Pasare., C et al.

### Edible Products

The edible product according to the present invention may be any edible product including food and beverage products and food supplements (which are intended to be taken as a supplement with other foods and not intended to be consumed as a food product per se). Examples of food supplements are vitamin and mineral supplements and the like. It is preferred according to the present invention that the edible product is a food or beverage product.

Different types of food products may be prepared according to the invention for example, meal replacers and other products to be used in a weight control programme, stews, noodles, ice-cream, sauces, dressings, seasonings, spreads such as margarine, snacks, cereals including cereal products such as porridges, beverages including fruit and/or vegetable containing beverages, sweet or savoury decorations, bread and bread products, biscuits and other bakery products, sweets, bar products, chocolate, chewing gum and dairy products. Different types of beverages may be prepared according to the invention for example, soups, ready-to-drink beverages and powdered beverages. The drinks may be protein based such as dairy or soy based products or may be soft drinks which are not based on protein.

Table 1 indicates a number of products, which may be prepared according to the invention, and a typical serving size thereof.

TABLE 1

| Product | Typical Serving size | |
|---|---|---|
| Margarine and other | 15 | g |
| spreads | | |
| Ice-cream and other frozen | 150 | g |
| confectionery products | | |
| Dressings and dips | 30 | g |
| Bar and snack products | 75 | g |
| including meal replacer | | |
| products | | |
| Meal replacer beverages | 330 | ml |
| Beverage shot products, | 100 | ml |
| including fruit and | | |
| vegetable based shot | | |
| products | | |
| Beverages (not meal | 200 | ml |
| replacer drinks or shot | | |
| drinks) | | |
| Biscuits | 20 | g |
| Yogurts and other dairy or | 150 | g |
| soy based desserts | | |

According to one embodiment wherein pH adjustment is used as one of the sub-lethal treatments, the present invention is especially suitable for preparing edible products which have a pH at which bacteria providing a health benefit are normally not stable. In particular the invention can be advantageously used for the preparation of edible products having a pH of 4 or less, for example from 3.8 to 2.0, more preferred 3.5 to 2.5, most preferred 3.3 to 2.8. Examples of such products are beverages, for example some soft drinks e.g. of the cola type or fruit/vegetable juices or fruit/vegetable based drinks such as lemon or orange juice.

Accordingly in another aspect the present invention relates to an edible product having a pH of 4 or less and made by the method of the invention.

Alternatively the invention can advantageously be used for the preparation of food products having a pH of 5.0 or more, for example from 5.0 to 10.0, more preferred 5.1 to 8.0, most preferred 5.2 to 7.0. Examples of such products are for example sauces, milk, margarines, bakery products, meal replacers, ice-cream etc.

The edible products may comprise a fermentation source. For example the food product of the invention may already be fermented before addition of the bacteria in accordance with the invention, such as brined vegetables or a variety of indigenous foods.

### Margarines and Other Spreads

Typically these are oil-in-water or water-in-oil emulsions, also spreads which are substantially fat free are covered. Typically these products are spreadable and not pourable at the temperature of use e.g. 2-10° C. Fat levels may vary within a wide range e.g. full fat margarines with 60-90 wt % of fat, medium fat margarines with 30-60 wt % of fat, low fat products with 10-30 wt % of fat and very low or fat free margarines with 0 to 10 wt % of fat.

The fat in the margarine or other spread may be any edible fat, often used are soybean oil, rapeseed oil, sunflower oil and palm oil. Fats may be used as such or in modified form e.g. hydrogenated, esterified, refined etc. Other suitable oils are well known in the art and may be selected as desired.

The pH of a margarine or spread may advantageously be from 5.0 to 6.5.

Examples of spreads other than margarines are cheese spreads, sweet spreads, yogurt spreads etc.

Optional further ingredients of spreads may be emulsifiers, colourants, vitamins, preservatives, emulsifiers, gums, thickeners etc. The balance of the product will normally be water.

A typical size for an average serving of margarine or other spreads is 15 grams.

### Frozen Confectionery Products

For the purpose of the invention the term frozen confectionery product includes milk containing frozen confections such as ice-cream, frozen yoghurt, sherbet, sorbet, ice milk and frozen custard, water-ices, granitas and frozen fruit purees.

Preferably the level of solids in the frozen confection (e.g. sugar, fat, flavouring etc) is more than 3 wt %, more preferred from 10 to 70wt, for example 40 to 70 wt %.

Ice-cream will typically comprise 2 to 20 wt % of fat, 0 to 20 wt % of sweeteners, 2 to 20 wt % of non-fat milk components and optional components such as emulsifiers, stabilisers, preservatives, flavouring ingredients, vitamins, minerals, etc, the balance being water. Typically ice-cream will be aerated e.g. to an overrun of 20 to 400%, more general 40 to 200% and frozen to a temperature of from −2 to −200° C., more general −10 to −30° C. Ice-cream normally comprises calcium at a level of about 0.1 wt %.

A typical size of an average serving of frozen confectionery material is 150 grams.

### Dressings and Dips

Generally dressings (including mayonnaise) or dips are oil-in-water emulsions. The oil phase of the emulsion generally comprise 0 to 80 wt % of the product. The level of fat is typically from 10 to 80% depending on the type of dressing or dip. Low or no fat dressings may for example contain triglyceride levels of 0, 5, 10, 15% by weight.

Dressings and dips are generally low pH products having a preferred pH of from 2-6.

Dressings or dips may optionally contain other ingredients such as emulsifiers (for example egg-yolk), stabilisers, acidifiers, biopolymers, bulking agents, flavours, colouring agents etc. The balance of the composition is water which could advantageously be present at a level of 0.1 to 99,9 wt %, more general 20-99 wt %, most preferred 50 to 98 wt %.

A typical size for an average serving of dressings or dips is 30 grams.

### Snacks and Bar Products Including Meal Replacer Snacks and Bars

These products often comprise a matrix of edible material wherein the bacteria can be incorporated. For example the matrix may be fat based (e.g. couverture or chocolate) or may be based on bakery products (bread, dough, cookies etc) or may be based on agglomerated particles (rice, grain, nuts, raisins, fruit particles).

Further ingredients may be added to the product such as flavouring materials, vitamins, minerals etc.

### Meal Replacer Beverages and Other Beverages (Including Beverage Shots)

The non-viable bacteria can advantageously be included in beverages for example soups, fruit and/or vegetable juices, soft drinks, dairy based drinks and soy based drinks etc. Advantageous beverages in accordance with the invention are tea based beverages and meal replacer beverages. These products will be described in more detail herein below. It will be apparent that similar levels and compositions apply to other beverages according to the invention.

For the purpose of this invention the term tea based products refers to products containing tea or tea replacing herbal compositions e.g. tea-bags, leaf tea, herbal tea bags, herbal infusions, powdered tea, powdered herbal tea, ice-tea, ice herbal tea, carbonated ice tea, carbonated herbal infusions etc.

Typically some tea based products of the invention may need a preparation step shortly before consuming, e.g. the making of tea brew from tea-bags, leaf tea, herbal tea bags or herbal infusions or the solubilisation of powdered tea or powdered herbal tea. For these products it is preferred to adjust the level of non-viable bacteria in the product such that one serving of the final product to be consumed has the desired levels of bacteria as described above.

For ice-tea, ice herbal tea, carbonated ice tea, carbonated herbal infusions the typical size of one serving will be 200 ml. Beverage shot products are beverages which are have a concentrated level of at least one active ingredient so that they deliver the full benefit of the active ingredient in a smaller volume of the beverage, thus they are generally provided in smaller quantities than other types of beverages as a single serving, a serving size of 100 ml is typical for a shots product.

Meal replacer drinks are typically based on a liquid base which may for example be thickened by means of gums or fibres and whereto a cocktails of minerals and vitamins are added. The drink can be flavoured to the desired taste e.g. fruit or choco flavour. A typical serving size may be 330 ml.

For products which are extracted to obtain the final product, generally the aim is to ensure that one serving comprises the desired amounts as indicated above. In this context it should be appreciated than normally only part of the non-viable bacteria present in the tea based product to be extracted will eventually be extracted into the final tea drink. To compensate for this effect generally it is desirable to incorporate into the products to be extracted about 2 times the amount as is desired to have in the extract.

For leaf tea or tea-bags typically 1-5 grams of tea would be used to prepare a single serving of 200 mls.

If tea-bags are used, the Lactobacillus may advantageously be incorporated into the tea component. However it will be appreciated that for some applications it may be advantageous to separate the non-viable bacteria from the tea, for example by incorporating it into a separate compartment of the tea bag or applying it onto the tea-bag paper.

### Biscuits

The biscuits according to the present invention may be of any type as desired. The non-viable bacteria according to the present invention may be included as a part of the biscuits themselves or as a decoration, coating or filling therefor. A typical serving size for a biscuit is 20 g.

### Yoghurt or and Other Dairy or Soy Based Desserts

The Yoghurt or and other dairy or soy based desserts according to the present invention may be of any type as desired. These products may be fermented by other bacteria than the non-viable bacteria according to the present invention. Alternatively, they may at least in part be fermented by the beneficial bacteria according to the invention before they are rendered non-viable. A typical serving size for these desserts is 150 g.

The invention will be further illustrated by reference to the following examples. Further examples within the scope of the invention will be apparent to the person skilled in the art.

### EXAMPLES

### Example 1

Lactobacillus reuteri SD2112 was cultivated in ‘Special MRS’ which was prepared by the following procedure. MRS (Merck, Germany) medium was acidified to pH 3.0 with concentrated HCl to precipitate proteins. This solution was stored overnight at 5° C. and centrifuged for 10 min at 5000 rpm. The supernatant was filtered using a 0.2 μm bottletop filter and the pH was adjusted to the original value of MRS (pH 5.7±0.2). This solution was filter sterilised using a 0.1 μm bottletop filter connected to a sterile bottle and stored (prior to use) at 5° C.

10 ml Special MRS was inoculated with 0.5% of a culture of L. reuteri SD2112 that has been stored at −80° C. as a fully grown culture in skim milk, diluted with sterile 10% glycerol to an end volume of 6% glycerol. L. reuteri SD2112 was pre-cultured overnight at 37° C. The final cultivation was performed in a 300 ml flask containing 250 ml Special MRS. The flask was inoculated with 5 ml of the pre-culture and incubated for 24 hours at 37° C.

After cultivation the medium was centrifuged in sterile Falcon tubes of 50 ml (5 min at 5000 rpm), the pellets were pooled in 1 tube and washed twice with a Peptone Physiological Salt (PPS, Tritium, The Netherlands, 0.1% peptone, 0.85% NaCl) solution. Subsequently the pellet was re-suspended in 5 ml PPS. This cell concentrate was used for further treatments.

Sterilised 5-ml glass tubes were filled with either 2.7 ml of PPS (or 2.7 ml of an acetic acid solution in case of a pH treatment). The acetic acid solution (HAc, pH3) was prepared by adding 13 μl acetic acid (100%) to 40 ml demineralised water. The pH of this solution was adjusted to pH 3 with concentrated HCl and filter sterilised using a 0.2 μm filter. The amount of undissociated acid in this solution is 0.3 g/l.

To the PPS (or HAc), 0.3 ml of the cell concentrate was added. Samples were mixed and subjected to different treatments as shown in Table 1.

TABLE 1

| | Step 1 | Step 2 | Step 3 |
|---|---|---|---|
| 1 | 65 min RT ⁽¹⁾ | — | — |
| 2 | 65 min HAc (pH 3) | — | — |
| 3 | 60 min RT | 5 min 60° C. ⁽²⁾ | — |
| 4 | 55 min RT | 10 min nisin ⁽³⁾ | — |
| 5 | 35 min RT | 30 min 100° C. ⁽²⁾ | — |
| 6 | 60 min HAc (pH 3) | 5 min 60° C. | — |
| 7 | 50 min RT | 5 min 60° C. | 10 min nisin |

⁽¹⁾ Room temperature
⁽²⁾ Samples that were subjected to heat were cooled down in melting ice for 2 minutes, before further treatment.
⁽³⁾ 30 μl Nisin was added from a freshly made nisin stock solution (100 ppm), prepared by dissolving 250 mg nisin (Sigma, Germany, 2.5%, porcine) in 50 ml PPS. The solution was sterilised using a 0.2 μm filter and stored at 5° C.

All samples were diluted in PPS directly after the treatment(s), up to 10⁻⁸ dilution for all except the 100° C. sample (up to 10⁻³ dilution). For all samples the 10⁻⁵ to 10⁻⁸ dilutions were put in a petridish (10⁻¹ to 10⁻³ for 100° C. sample) and MRS agar of 50° C. was added to the plates (poring method). After coagulation of the agar the plates were incubated anaerobically at 37° C. for at least 2 days.

The 10⁻³ dilution (in PPS) of all treatments was used for flowcytometric measurements. 1 ml of each of these 10⁻³ samples was added to 5 pl of Propidium iodide (PI) in a 4 ml sterile plastic tube, mixed and incubated for 5 minutes before the flowcytrometric measurement was performed. PI will only enter leak cells and therefore is a measure for the damage the probiotics have taken. The results of the plating, the amount of living cells in Colony Forming Units per ml, and the percentage of leak cells is given in Table 2.

TABLE 2

| | | Viable Count | PI stained |
|---|---|---|---|
| | Sample | (CFU/ml) | (%) |
| 1 | Untreated | 3.0 · 10⁹ | 0.5 |
| 2 | 30 min 100° C. | ~10³ | 97 |
| 3 | Control 60° C. | 2.6 · 10⁹ | 0.9 |
| 4 | Control pH3 | 2.8 · 10⁹ | 0.6 |
| 5 | Control 1 ppm nisin | 0.9 · 10⁹ | 40 |
| 6 | pH 3 + 60° C. | 1.3 · 10⁸ | 40 |
| 7 | 60° C. + 1 ppm nisin | <1 · 10⁴ | 58 |

From these results it is concluded that only cells that have been subjected to 2 sub-lethal treatments, are capable of absorbing significant quantities of propidiumiodide. Furthermore, the results show that a combination of two sub-lethal treatments can render the viable bacteria non-viable even if each sub-lethal treatment itself only had limited impact on viability.

### Example 2

Probiotic bacteria of selected strains (e.g. Lactobacillus reuteri SD2112, L. rhamnosus HN001, L plantarum WCFS1, L. delbrueckii LMG6891, L casei immunitas, Bifidobacterium lactis Bb-12) can be exposed to two or more of various sub-lethal treatments which in combination render them non-viable without loosing all of their probiotic characteristics.

Viable probiotic bacteria at a concentration of 10⁶-10⁸ cfu/ml can;
1) remained untreated (positive control), or
2) be incubated at 100° C. for 30 min (negative control),or
3) be exposed to the following combinations of sub-lethal treatments;
3.1 heating to a temperature of 150° C. above the optimal growing temperature for 5 minutes, followed directly by adding at least one of between 0.1 and 1 ppm lysozym, between 0.9 mM and 5 mM sodium sorbate or between 0.05 and 1 ppm nisin, or
3.2 heating to a temperature of 15° C. above the optimal growing temperature for 5 minutes, followed directly by applying a pressure of from 150 Mpa to 400 Mpa at 5° C. for 20 to 60 seconds, or
3.3. heating to a temperature of 15° C. above the optimal growing temperature for 5 minutes, followed directly by applying a pulsed electric field using between 5 and 100 kV/cm with a pulse length of between 1 and 10 μs at 10° C., or
3.4 adding at least one of between 0.1 and 1 ppm lysozym, between 0.9 mM and 5 mM sodium sorbate or between 0.05 and 1 ppm nisin, followed directly by applying a pressure of from between 150 Mpa to 400 Mpa at 5° C. for between 20 and 60 seconds, or
3.5 adding at least one of between 0.1 and 1 ppm lysozym, between 0.9 mM and 5 mM sodium sorbate or between 0.05 and 1 ppm nisin, followed directly by applying a pulsed electric field using between 15 and 100 kV/cm with a pulse length of between 1 and 10 μs at 10° C.

Following any of the two sub-lethal treatment steps combinations 3.1 to 3.5 above and the control treatments 1 and 2, the probiotic bacteria can be counted by serial dilution in a suitable dilution medium, followed by plating on selective agar medium under anaerobic conditions for about 24 hours at 37° C., to assess the residual number of colony forming units and thus verify efficiency of rendering the probiotic bacteria non-viable. Other aliquots can be used to assess the integrity of the DNA, e.g. by agarose gel electrophoresis or by other suitable methods known to those skilled in the art and/or aliquots can be used to compare the immunomodulating activity of the different probiotic bacteria preparations.

One example of assessing immunomodulating activity is to incubate peripheral blood mononuclear cells (PBMC) derived from the blood of human volunteers for various times (24-48 hour) with serial dilutions of the different probiotic bacteria preparations, or with 0.1-10 μg/mL of DNA isolated from the various probiotic bacteria preparations. The signaling events triggered by the interaction of these preparations with the freshly isolated human PBMC can be assessed as activation of various kinases, translocation of NFKB or by the resulting downstream effects such as cytokine production.

Whereas it is recognised that sensitivity of the different probiotic bacteria strains for distinct combinations of sub-lethal treatments may vary, the general teaching is that heat treated probiotic bacteria (treatment 2 above) and probiotic bacteria treated according to the invention (treatments 3.1 to 3.5 above) are rendered non-viable. Furthermore, the untreated probiotic bacteria (treatment 1) and the probiotic bacteria treated according to the invention (treatments 3.1 to 3.5 above) retain at least some of the modulating effect on the activity of human PBMC. This indicates that probiotic bacteria subjected to the sub-lethal treatments according to the invention (treatments 3.1 to 3.5 above) may exert beneficial probiotic effects when administered in the context of a food product without bringing all the problems associated with the use of live probiotic bacteria. However, this is not the case when the bacteria are rendered non-viable by lethal treatments such as a conventional heat treatment (treatment 2).

### Example 3

Preparations of probiotic bacteria strains prepared as described in example 2 treatments 3.1 to 3.5 above, can advantageously be post-added to a concentrate beverage based on fruit and vegetable extracts or based on soy protein (a so-called ‘shot’ product) to add the probiotic benefits to the shot product without altering organoleptic and taste properties thereof by post-acidification by the probiotics.

1. A method of preparing an edible product comprising non-viable bacteria providing a health benefit to the subject consuming the bacteria, wherein the method comprises subjecting viable bacteria to at least two sub-lethal treatments to obtain the non-viable bacteria providing a health benefit, each sub-lethal treatment on its own not being sufficient to render the bacteria non-viable.

2. A method according to claim 1, comprising subjecting the viable bacteria to at least two sub-lethal treatments, at least one of which sub-lethal treatments reduces the replication capacity of the viable bacteria by at least 5%, preferably by at least 10%.

3. A method according to claim 1, comprising subjecting the viable bacteria to at least two sub-lethal treatments, wherein the sum of the percentages reduction in replication capacity observed for each sub-lethal treatment does not exceed 60%.

4. A method according to claim 1, comprising either;
a) subjecting viable bacteria providing said health benefit to at least two sub-lethal treatments and subsequently contacting the non-viable bacteria thereby produced with an edible product or at least one ingredient thereof, or
b) contacting viable bacteria providing said health benefit with an edible product and subsequently subjecting the edible product comprising the viable bacteria to at least two sub-lethal treatments, or
c) contacting viable bacteria providing said health benefit with at least one ingredient of an edible product and subsequently subjecting the mixture of the viable bacteria and the ingredient to at least two sub-lethal treatments.

5. A method according to claim 1, wherein the edible product is a food or beverage product.

6. A method according to claim 1, wherein the health benefit is a probiotic effect.

7. A method according to claim 1, wherein the bacteria are non-pathogenic bacteria.

8. A method according to claim 1, wherein the bacteria are substantially structurally intact in the edible product.

9. A method according to claim 8, wherein the bacteria retain conserved microbial patterns that can be recognized by pattern recognition receptors of the immune system.

10. A method according to claim 9, wherein the conserved microbial patterns comprise DNA and/or cell wall constituents.

11. A method according claim 1, wherein the bacteria are selected from the genera Lactobacillus or Bifidobacterium.

12. A method according to claim 1, wherein the edible product contains between 10⁶ and 10¹¹ bacteria per serving.

13. A method according to claim 1, wherein each of the two or more sub-lethal treatment steps is independently selected from;
(i) the application of pressure
(ii) adjusting the pH
(iii) adjusting the osmotic pressure
(iv) heating
(v) homogenisation
(vi) freeze-thaw cycles
(vii) spray-drying
(viii) adding one or more agents having a bactericidal effect
(ix) applying a pulsed electric field.

14. A method according to claim 13, each of the two or more sub-lethal treatment steps is independently selected from;
(i) Applying a pressure of from 150 Mpa to 400 Mpa at from −30° C. to 25° C. for between 20 to 60 seconds,
(ii) Adjusting the pH to in the range of from pH 3 to 5 or from pH8 to 9,
(iii) Adjusting the osmotic pressure by adding a suitable amount of an alkaline or alkaline earth metal salt,
(iv) Heating to a temperature of from 10° C. to 25° C. above the optimal growing temperature for the bacteria for between 1 to 5 minutes
(v) Homogenising at from 20 to 30 bar at 10° C. to 15° C. above the optimal growing temperature for the bacteria for between 1 to 5 minutes
(vi) Subjecting to a freezing step and subsequent thawing step for between 5 to 25 cycles.
(vii) Adding a suitable amount of one or more agent(s) that have a bactericidal effect and which are chosen from sodium sorbate, lysozym and nisin.
(viii) Applying a pulsed electric field using between 15 to 100 kV/cm with a pulse length of between 1 to 10 μs at from 10° C. to 50° C.

15. An edible product obtainable according claim 1.

16. An edible product according to claim 15, wherein said product is a food or beverage product.
